(19) United States
(12) Patent Application Publication
(10) Pub. No.: US 2007/0232994 A1
(43) Pub. Date: Oct. 4, 2007

(54) Needle-free injection device

(75) Inventors: Yutaro Sonoda, Kanagawa (JP); Atsushi Matsumoto, Kanagawa (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Tokyo (JP)

(21) Appl. No.: 11/730,293

(22) Filed: Mar. 30, 2007

(30) Foreign Application Priority Data
Mar. 30, 2006 (JP) 2006-095603

(51) Int. Cl. A61M 5/30

(52) U.S. Cl. 604/68

(57) ABSTRACT

The present invention relates to a needle-free injection device which includes a skin deforming member for deforming a skin, and a needle-free injector ejecting liquid by high pressure with respect to the skin deformed by the aforesaid skin deforming member and injecting the aforesaid liquid into the aforesaid skin.

### CROSS REFERENCES TO RELATED APPLICATIONS

The present invention contains subject manner related to Japanese Patent Application JP 2006-095603 filed in the Japanese Patent Office on Mar. 30, 2006, the entire contents of which being incorporated herein by reference.

### BACKGROUND OF THE INVENTION

1. Field of the Invention

The present invention relates to a needle-free injection device which makes a medical agent to go inside from a surface of a skin and to be injected into a predetermined region (for example, dermis).

2. Description of the Related Art

It is known that the dermis has a high density of capillary blood vessel when compared with an epidermis or a subcutis and also a lymph vessel end exists therein, so that in particular, a medical agent injected thereto directly is shifted to a blood vessel or a lymph vessel and an absorption speed thereof being absorbed in the body fluid is speedy. In particular, it is possible in the dermis to make a medical agent using a macromolecular substance such as hormone, antibody drug, cytokine and the like to be absorbed into the blood efficiently. Also, it is known that the dermis is a place of efficient immunity and it is possible to make a saving of applied dose of vaccine or to strengthen sensitization of a weak vaccine.

Also, it is known for adult human beings that the dermis exists approximately in a certain amount of depth from the body surface (surface of stratum corneum). In other words, this fact means, in case of injecting a medical agent into the dermis, that it is enough if the medical agent is to be injected approximately into a certain amount of depth from the body surface with respect to these human beings.

Generally, the width of the dermis is around 1 mm to 4 mm (average value is 1 mm to 2 mm) if the vertical direction with respect to the body surface is made to be a reference and also, as shown in the cross-section view of a general skin structure of FIG. 26, the dermis D exists in the skin so as to be sandwiched between an epidermis E which includes a stratum corneum SC and has width of around 0.06 mm to 0.1 mm and a subcutis S.

Accordingly, it is difficult to inject a medical agent certainly into the dermis which exists between the epidermis and the subcutis and if the medical agent is injected erroneously into the subcutis or the like, there occurs a problem such that a medical agent cannot be absorbed into the blood efficiently.

In recent years, for example, it is attempted that the macromolecule medicine mentioned above is administered continually or by one-shot into the dermis as a target and in such a case, in particular, the above-mentioned problem becomes conspicuous.

Here, a medicinal solution injection device is known in which the depth (insertion depth) of the puncture needle to be inserted into the skin is defined to be a predetermined length and the puncture needle is inserted into the skin from the vertical direction with respect to the body surface in order to inject a medical agent to a specific layer existing in the skin (Patent Document 1). In addition, there is known a needle-free hypodermic injector in which a nozzle having a minute opening hole is set vertically with respect to a body surface and a medical agent is jetted from the nozzle thereof by high pressure so as to inject it into the skin (see Patent Reference 2).

[Patent Document 1] Japanese Patent Laid-open No. 2005-87519

[Patent Document 2] Japanese Patent Laid-open No. 63-315063

### SUMMARY OF THE INVENTION

However, there is employed, in the medicinal solution injection device described in the Patent Document 1, a constitution in which the puncture needle is inserted into the skin from the vertical direction with respect to the body surface. In this case, if it is attempted to stick the puncture needle, the whole skin is sunk in elastically so as not to be punctured and also, even if it is punctured, it sometimes happens that the needlepoint cannot reach the dermis.

Also, when the puncture needle is inserted perpendicularly with respect to the dermis, the depth (insertion depth) of the puncture needle in the dermis becomes short and, for example, in such a case where some kind of impact or the like is added from the outside, there occurs a problem in which the puncture needle during injecting a medical agent drops out from the dermis.

Further, in case of using the medicinal solution injection device described in the Patent Document 1, the distance from an insertion aperture of the puncture needle which is formed on the surface of the dermis (boundary portion of epidermis and dermis) until a medical agent releasing opening which exists at the tip of the puncture needle becomes short in the dermis in which there happens a problem that the medical agent injected to the dermis from the medical agent releasing opening is to leaks from the insertion aperture to the outside of the dermis (epidermis)

Also, in needle-free hypodermic injector described in the Patent Document 2, there is employed a constitution in which the medical agent jetted from the nozzle by high pressure is to go into in the vertical direction with respect to a layer of a skin such as epidermis, dermis or the like. In this case, when the medical agent is injected into the dermis, there occurs a problem in which it is difficult to inject the medical agent efficiently, because the dermis is a thin layer.

In consideration of the above-mentioned problems, an object of the present invention lies in providing a needle-free injection device which can inject a medical agent into a predetermined layer of a skin easily and certainly.

Such an object is accomplished according to the present invention of the following (1) to (12).
(1) A needle-free injection device comprising:

a skin deforming member for deforming a skin; and

a needle-free injector ejecting liquid by high pressure with respect to the skin deformed by aforesaid skin deforming member and injecting aforesaid liquid into aforesaid skin.
(2) The needle-free injection device described in aforesaid (1), wherein aforesaid skin deforming member has two or more planes fixed on the skin and the skin is deformed so as to form a step portion by aforesaid two or more planes.
(3) The needle-free injection device described in aforesaid (2), wherein it is constituted such that aforesaid needle-free injector ejects aforesaid liquid approximately parallel to the any one of aforesaid two or more planes of aforesaid skin deforming member and injects it into aforesaid step portion.
(4) A needle-free injection device comprising:

a needle-free injector ejecting filled-up liquid by high pressure;

a base member supporting aforesaid needle-free injector to be movable;

a skin deforming member mounted on aforesaid base member and also deforming the skin, wherein

it is constituted such that aforesaid needle-free injector is moved so as to approach to the skin deformed by aforesaid skin deforming member and the liquid is ejected by high pressure and injected with respect to aforesaid deformed skin.
(5) The needle-free injection device described in aforesaid (4), wherein aforesaid needle-free injector comprises a slide member supported on aforesaid base portion to be movable, and an injector body mounted on aforesaid slide member detachably.
(6) The needle-free injection device described in aforesaid (4), wherein aforesaid skin deforming member comprises:

a first tabular portion supported on aforesaid base member to be revolvable and also having a plane fixed on the skin;

a coupling portion connected to aforesaid first tabular portion through a first hinge portion and also having a plane fixed on the skin; and

a second tabular portion connected to aforesaid coupling portion through a second hinge portion and also having a plane fixed on the skin, wherein

the skin is deformed so as to form a step portion between aforesaid first tabular portion and aforesaid second tabular portion by bending-deforming aforesaid skin deforming member.
(7) The needle-free injection device described in aforesaid (6), wherein aforesaid first hinge portion is provided by forming a V-shaped groove to be opened downward at a connection portion of aforesaid first tabular portion and aforesaid coupling portion and also, aforesaid second hinge portion is provided by forming a V-shaped groove to be opened upward at a connection portion of aforesaid second tabular portion and aforesaid coupling portion.
(8) The needle-free injection device described in aforesaid (6), wherein aforesaid skin deforming member is bending-deformed by depressing aforesaid second tabular portion to aforesaid first tabular portion side so as to turn aforesaid first tabular portion upward.
(9) The needle-free injection device described in aforesaid (6), wherein there is provided with a spring member biasing aforesaid first tabular portion upward and aforesaid first tabular portion is turned by the spring force of aforesaid spring member so as to bending-deform aforesaid skin deforming member.
(10) The needle-free injection device described in aforesaid (6), wherein there is provided with a magnet portion on either one of aforesaid first tabular portion or aforesaid base member and also there is provided with a magnetic portion on the other one thereof which pulls aforesaid magnet portion toward each other, and aforesaid first tabular portion is made to turn by the magnetic force operated between aforesaid magnet portion and aforesaid magnetic portion so as to bending-deform aforesaid skin deforming member.
(11) A needle-free injection device comprising

a positioning portion having a plane fixed on a skin;

a coupling portion connected to aforesaid positioning portion to be revolvable through a hinge portion and also having a plane fixed on the skin; and

a needle-free injector constituted integrally with aforesaid coupling portion and also being provided with an ejecting opening on a plane fixed on the skin of aforesaid coupling portion, wherein

it is constituted such that the skin fixed on aforesaid positioning portion and aforesaid coupling portion is uplifted by changing posture of aforesaid needle-free injector and by turning aforesaid coupling portion with respect to aforesaid positioning portion, and liquid is ejected from aforesaid ejecting opening by high pressure and injected into aforesaid uplifted skin.
(12) The needle-free injection device described in aforesaid (11), wherein when aforesaid coupling portion is turned with respect to aforesaid positioning portion, the plane fixed on the skin at aforesaid coupling portion becomes approximately perpendicular with respect to the plane fixed on the skin of aforesaid positioning portion.
(13) The needle-free injection device described in aforesaid (11), wherein there is provided with an engagement projection portion on one of aforesaid positioning portion and aforesaid needle-free injector and also there is provided with an engagement concave portion on the other one thereof which is to be engaged with aforesaid engagement projection portion when aforesaid coupling portion is turned with respect to aforesaid positioning portion, and it is constituted such that a state in which aforesaid coupling portion is turned with respect to aforesaid positioning portion is to be maintained.

According to the present invention, it is possible to inject a medical agent easily and certainly with respect to a predetermined region of a skin.

### BRIEF DESCRIPTION OF THE DRAWINGS

FIG. 1 is an external perspective view showing a first exemplified embodiment of a needle-free injection device of the present invention;

FIG. 2 is an exploded perspective view of the needle-free injection device shown in FIG. 1;

FIG. 3 is a cross-section view of a base member shown in FIG. 2 by A-A line;

FIG. 4 is an explanatory diagram, in a state in which the skin deforming member of the needle-free injection device shown in FIG. 1 is bending-deformed, seeing it from the right side surface by assuming that a bearing portion 311 side is to be front face;

FIG. 5 is an explanatory diagram showing an adhesion member to be mounted on the skin deforming member of the needle-free injection device shown in FIG. 1;

FIG. 6 is a perspective view seeing the needle-free injector of the needle-free injection device shown in FIG. 1 from the front face side;

FIG. 7 is a cross-section view indicating the inside of needle-free injector shown in FIG. 6 schematically;

FIGS. 8A to 8C are cross-section views for explaining the usage of the needle-free injection device shown in FIG. 1;

FIG. 9 is an external perspective view indicating an embodiment in which there is provided with a cover member covering the nozzle of the needle-free injector for the needle-free injection device shown in FIG. 1;

FIG. 10 is an external perspective view showing a second exemplified embodiment of a needle-free injection device of the present invention.

FIG. 11 is an exploded perspective view of the needle-free injection device shown in FIG. 10;

FIG. 12 is an explanatory diagram indicating a mounting state of the spring member of the needle-free injection device shown in FIG. 10;

FIG. 13 is an explanatory diagram showing another example of the spring member used for the needle-free injection device shown in FIG. 10;

FIGS. 14A to 14C are cross-section views for explaining the usage of the needle-free injection device shown in FIG. 10;

FIGS. 15A to 15C show a third exemplified embodiment of a needle-free injection device of the present invention and are cross-section views for explaining the usage thereof;

FIG. 16 is an external perspective view showing a fourth exemplified embodiment of a needle-free injection device of the present invention;

FIG. 17 is a cross-section view indicating the inside of the injector body of the needle-free injection device shown in FIG. 16 schematically;

FIG. 18 shows a fifth exemplified embodiment of a needle-free injection device of the present invention and is a side elevational view thereof;

FIG. 19 is an enlarged view enlarging the positioning portion, the needle-free injector and the like of the needle-free injection device shown in FIG. 18;

FIG. 20 is an explanatory diagram seeing the positioning portion, the needle-free injector and the like of the needle-free injection device shown in FIG. 18 from the lower side;

FIG. 21 is an explanatory diagram for explaining the positioning portion of the needle-free injection device shown in FIG. 18;

FIG. 22 is a side elevational view showing a state in which the needle-free injector of the needle-free injection device shown in FIG. 18 is turned by approximately 90 degree with respect to the positioning portion;

FIG. 23 is an explanatory diagram seeing the positioning portion, the needle-free injector and the like in a state shown in FIG. 22 from the lower side;

FIG. 24 explains the usage of the needle-free injection device shown in FIG. 18 and is a cross-section view showing a state in which the positioning portion and the interlinked protrusion portion are bonded on the body surface;

FIG. 25 explains the usage of the needle-free injection device shown in FIG. 18 and is a cross-section view showing a state in which the skin is uplifted by turning the interlinked protrusion portion approximately by 90 degree with respect to the positioning portion; and

FIG. 26 is a cross-section view showing a general skin structure.

### DESCRIPTION OF THE PREFERRED EMBODIMENTS

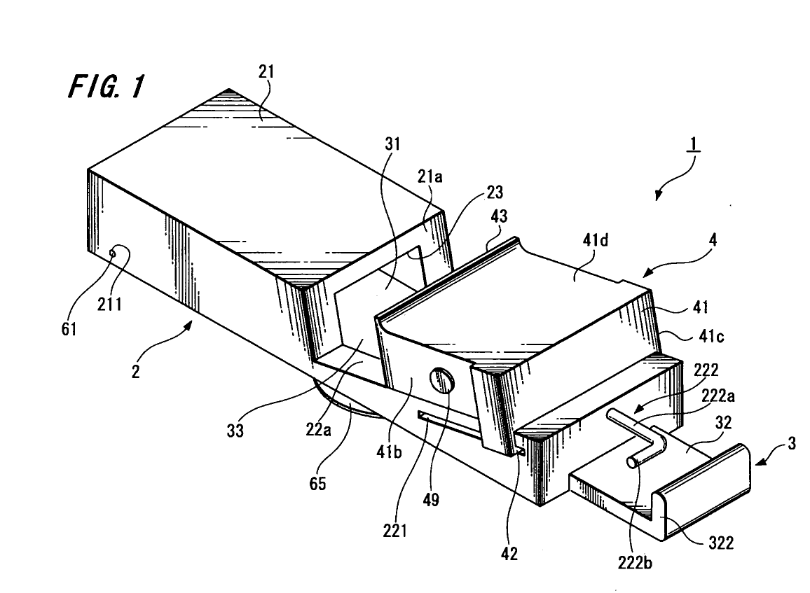

Hereinafter, it will be explained with respect to the best mode for practicing the needle-free injection device of the present invention with reference to FIG. 1 to FIG. 25, wherein it should be noted that the present invention is not limited by the following modes.

First, it will be explained with respect to a first exemplified embodiment of a needle-free injection device of the present invention.

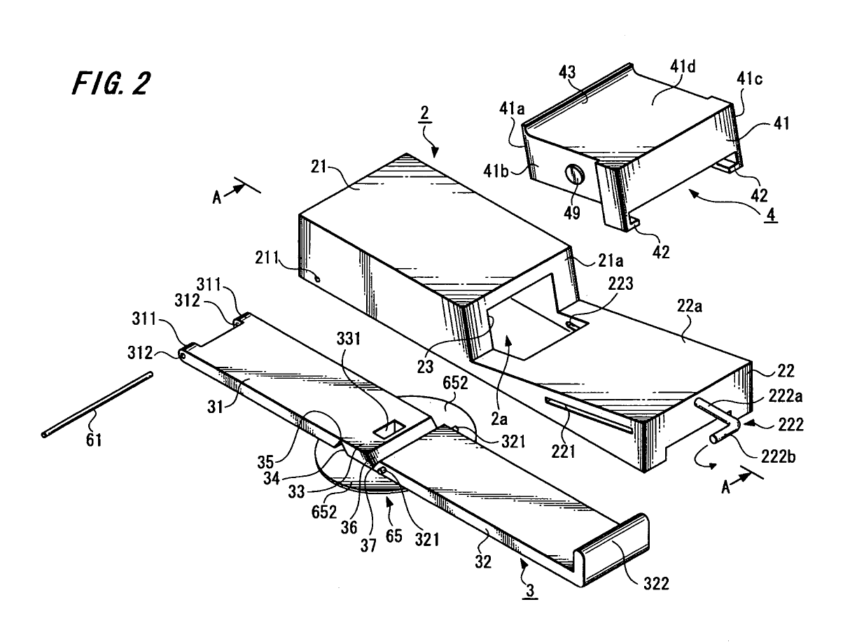

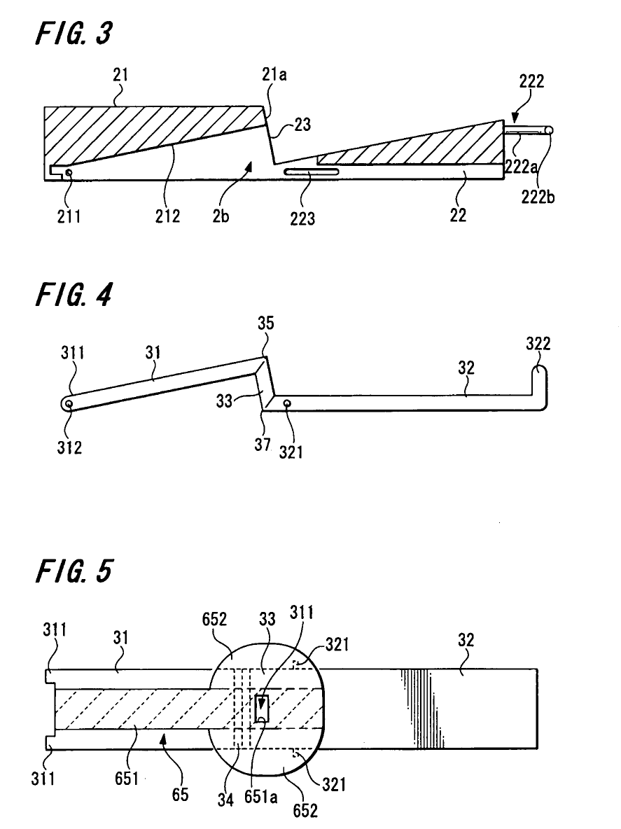

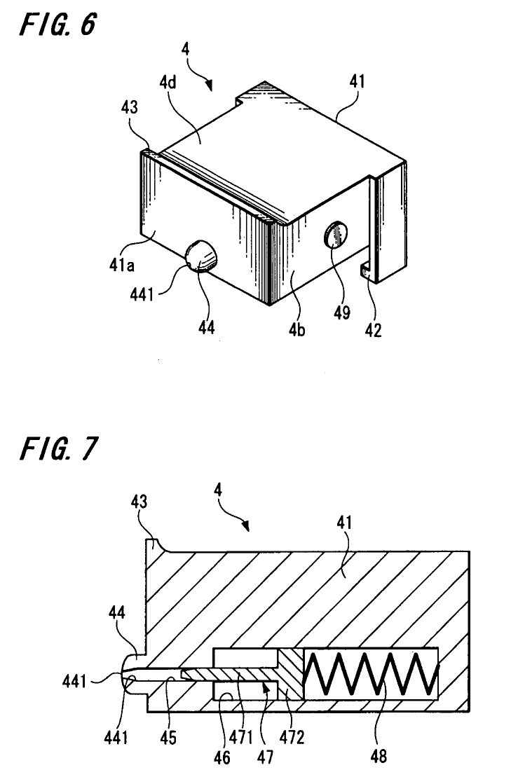

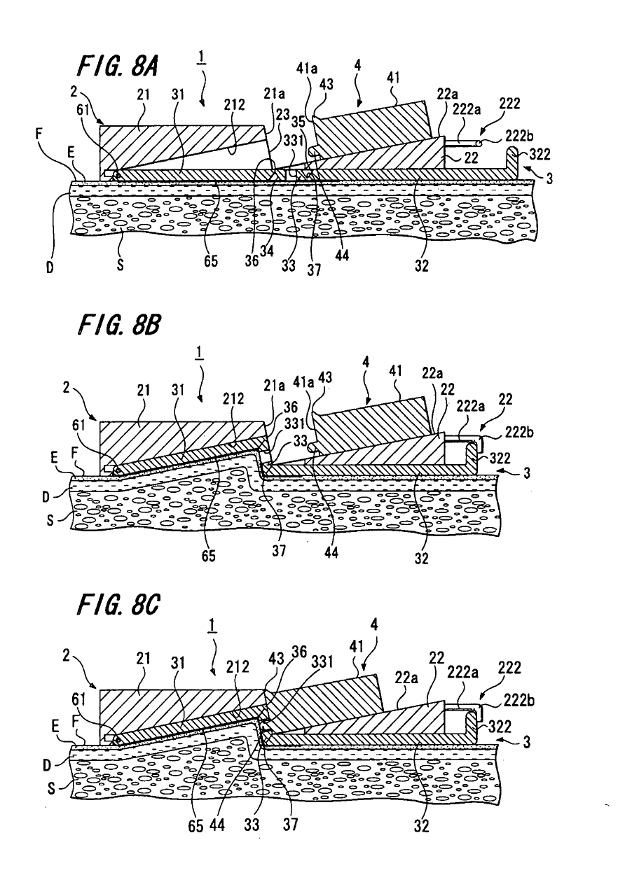

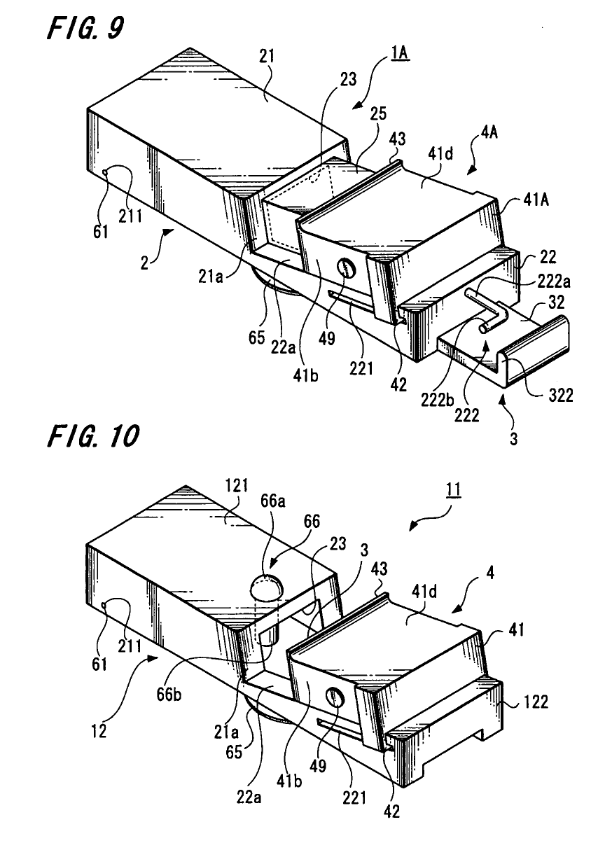

FIG. 1 to FIG. 8 show a first exemplified embodiment of a needle-free injection device of the present invention, wherein FIG. 1 is an external perspective view, FIG. 2 is an exploded perspective view, FIG. 3 is a cross-section view by A-A line of the base member shown in FIG. 2, FIG. 4 is an explanatory diagram showing a state in which the skin deforming member is bending-deformed, FIG. 5 is an explanatory diagram showing an adhesion member mounted on the skin deforming member, FIG. 6 is a perspective view of the needle-free injector, FIG. 7 is a cross-section view indicating the inside of the needle-free injector shown in FIG. 6 schematically, and FIGS. 8A to 8C are cross-section views for explaining the usage of the needle-free injection device shown in FIG. 1. Also, FIG. 9 is an external perspective view indicating a modified embodiment of the needle-free injection device shown in FIG. 1.

As shown in FIG. 1 and FIG. 2, a needle-free injection device 1 showing a first exemplified embodiment of a needle-free injection device of the present invention is constituted by including a base member 2, a skin deforming member 3 mounted on the base member 2 revolvably, a needle-free injector 4 supported on the base member 2 to be movable slidably and the like. It should be noted that a stratum corneum SC, an epidermis E including the stratum corneum SC, a dermis D, and a subcutis S are arranged in a form of laminae on the lower side of the body surface F on which the skin deforming member 3 is fixed (see FIGS. 8A to 8C or the like).

The base member 2 of the needle-free injection device 1 is formed to be totally an elongated housing and is constituted by a turning control portion 21 on which the skin deforming member 3 is mounted revolvably, a support board portion 22 supporting the needle-free injector 4 to be movable leading to this turning control portion 21 and the like. There is provided on this base member 2 with a storing portion 2a which is extended continuously to the longitudinal direction of the turning control portion 21 and the support board portion 22, and this storing portion 2a is opened to the lower surface side.

The turning control portion 21 of the base member 2 is formed to be a horizontally long housing, a side surface 21a of one of the short sides is made to be continuous with the support board portion 22. There are provided on both the side surfaces of the long sides of this turning control portion 21 with bearing openings 211, 211 (only one-half thereof is shown in FIG. 2 or the like) passing therethrough in the horizontal direction. The bearing openings 211, 211 of the turning control portion 21 are arranged at the lower portions on the opposite side of the support board portion 22 so as to face to each other and a hinge axis 61 is inserted through these bearing openings 211, 211.

As shown in FIG. 3, there is provided in the inside of the turning control portion 21 constituting a portion of the storing portion 2a with a latch slant face 212 showing one embodiment of a latch portion latching the turn of a first tabular portion 31 of the skin deforming member 3, which will be described later. Also, the side surface 21a which is continuous with the support board portion 22 of the turning control portion 21 is inclined so as to be orthogonal with respect to the latch slant face 212 and the needle-free injector 4 is contacted to this side surface 21a.

As shown in FIG. 2 and the like, the support board portion 22 of the base member 2 is formed to be a horizontally long housing similarly as the turning control portion 21 and the upper surface 22a thereof is made to be a slant face in parallel with the latch slant face 212 of the turning control portion 21. The upper surface 22a of this support board portion 22 is made to be orthogonally continuous with the side surface 21a of the turning control portion 21. Then, there is provided with the opening window 23 from the side surface 21a of the turning control portion 21 until the upper surface 22a of the support board portion 22 and a nozzle 44 of the needle-free injector 4 to be described later is to be inserted into this opening window 23.

There are provided on both the side surfaces of the long sides of the support board portion 22 with first guide grooves 221, 221 (only one-half thereof is shown in FIG. 2 and the like) for guiding the movement of the needle-free injector 4. These first guide grooves 221, 221 are extended by being inclined so as to become in parallel with the upper surface 22a of the support board portion 22 and they are constituted so as to guide the needle-free injector 4 along the upper surface 22a of the support board portion 22.

Also, there is provided, on the side surface of the opposite side with respect to the turning control portion 21 of the support board portion 22, with a lock pin 222 latching the movement of the second tabular portion 32 of the skin deforming member 3, which will be described later. This lock pin 222 forms approximately an L-shape and is formed by an axis portion 222a and a latch arm 222b which is continuous with this axis portion 222a.

The axis portion 222a of the lock pin 222 is mounted on the support board portion 22 rotatably and also, is made to be possible to be fixed thereon in an arbitrary position. By rotating this axis portion 222a and directing the tip of the latch arm 222b downward, the latch arm 222b is made to be engaged with the second tabular portion 32 of the skin deforming member 3.

As shown in FIG. 3 and the like, there are provided, in the inside of the support board portion 22 which constitutes a portion of the storing portion 2a, with second guide grooves 223, 223 (only one-half thereof is shown in FIG. 3 and the like) for guiding the movement of the second tabular portion 32 of the skin deforming member 3. These second guide grooves 223, 223 are extended to the horizontal direction and they are constituted such that the second tabular portion 32 of the skin deforming member 3 will be guided in the horizontal direction owing to that configuration.

The skin deforming member 3 which is arranged on the storing portion 2a of the base member 2 is made to be a plate body forming an elongated rectangle and constituted by the first tabular portion 31 mounted on the turning control portion 21 to be revolvable, the second tabular portion 32 guided by the second guide grooves 223, 223 of the support board portion 22, a coupling portion 33 interlinking the first tabular portion 31 and the second tabular portion 32 and the like.

There are provided with a pair of bearing pieces 311, 311 at the edge portion on the opposite side with respect to the coupling portion 33 of the first tabular portion 31 and there are provided at these bearing pieces 311, 311 with bearing openings 312, 312 which pass therethrough in the horizontal direction. Then, by inserting the hinge axis 61 into the respective bearing openings 312, 312 of the first tabular portion 31 and into the respective bearing opening 211, 211 of the turning control portion 21, the first tabular portion 31 and the turning control portion 21 are hinge-coupled and the first tabular portion 31 is mounted on the turning control portion 21 to be revolvable.

There are provided with guide pins 321, 321 on the coupling portion 33 side of both the side surfaces of the long sides of the second tabular portion 32. These guide pins 321, 321 will be engaged with the second guide grooves 223, 223 which are provided at the support board portion 22 of the base member 2 to be slidable, respectively. Thereby, the second tabular portion 32 is guided by the second guide grooves 223, 223 and made to be movable in the horizontal direction.

It should be noted that the guide pins 321, 321 may be constituted so as to be rotatable centering around the axis center thereof. In this case, it is possible to reduce friction resistance between the guide pins 321, 321 and the second guide grooves 223, 223 and it is possible to move the second tabular portion 32 more smoothly.

Also, there is provided with an operation piece 322 at the edge portion on the opposite side with respect to the coupling portion 33 of the second tabular portion 32. This operation piece 322 is provided in order to make the operation to become easy by being hooked with fingers when the second tabular portion 32 is moved to the horizontal direction, and it is formed so as to project upward continuously from the short side of the second tabular portion 32. Also, the operation piece 322 is to be engaged with the latch arm 222b of the lock pin 222 provided on the base member 2 when the second tabular portion 32 is moved toward the turning control portion 31 side, whereby the movement of the second tabular portion 32 is latched.

There is provided with a V-shaped groove 34 opened downward at the coupling portion between the first tabular portion 31 and the coupling portion 33, whereby a first hinge portion 35 is formed. Also, there is provided with a V-shaped groove 36 opened upward at the coupling portion between the second tabular portion 32 and the coupling portion 33, whereby a second hinge portion 37 is formed.

The first hinge portion 35 and the second hinge portion 37 are set to have low stiffness compared with that of each of the tabular portions 31, 32. For this reason, the skin deforming member 3 is made to be bending-deformable at the first hinge portion 35 and the second hinge portion 37. More specifically, as shown in FIG. 4, the skin deforming member 3 is constituted such that the coupling portion 33 is bendable so as to rise by being inclined with respect to the second tabular portion 32 of a state in parallel with the horizontal direction and at the same time, the coupling portion 33 is bendable so that the first tabular portion 31 becomes approximately perpendicular with respect to the coupling portion 33. In this case, the coupling portion 33 is inclined such that the planes thereof (upper surface and lower surface) become in parallel with the side surface 21a of the turning control portion 21 and at the same time, the first tabular portion 31 is inclined such that the planes thereof (upper surface and lower surface) become in parallel with the latch slant face 212 of the turning control portion 21.

Also, the coupling portion 33 is provided with a through-hole 331 which passed through the upper and lower surfaces thereof. This through-hole 331 is made to face to the nozzle 44 of the needle-free injector 4 when the skin deforming member 3 is bending-deformed. Then, by sliding the needle-free injector 4, the nozzle 44 thereof is inserted into the through-hole 331.

As shown in FIG. 5, an adhesive film 65 showing one embodiment of the adhesion member for adhesively fixing the skin deforming member 3 on the body surface F is glued on the lower surface of the skin deforming member 3. This adhesive film 65 is formed by a glue portion 651 forming an elongated rectangle and wing portions 652, 652 continuous with the long sides of this glue portion 651.

The glue portion 651 of the adhesive film 65 is set to have a length in the width direction shorter than the length in the width direction of the skin deforming member 3 and the length in the longitudinal direction thereof is set to have a length corresponding to the length from the tip portion on the first tabular portion 31 side of the skin deforming member 3 to the midway portion of the second tabular portion 32 through the coupling portion 33. There is provided with an opening hole 651a at the position facing to the through-hole 331 of the skin deforming member 3 of the glue portion 651 and it is constituted such that the body surface F is to be exposed from the through-hole 331 when the skin deforming member 3 is bonded on the body surface F.

It is constituted such that this adhesive film 65 is glued on the skin deforming member 3 by the whole surface of the glue portion 651 and at the same time is bonded to the body surface F by the whole surfaces of the glue portion 651 and wing portions 652, 652. More specifically, the adhesive film 65 has a constitution in which an adhesion surfaces are not provided on the faces of the skin deforming member 3 sides of the wing portions 652, 652.

It is possible for the substance of the adhesive film 65 to cite polyethylene terephthalate (PET) and polyethylene which do not expand or contract and the like, but it is not limited by this and it is possible to use polyurethane and others of various kinds of materials which are used as this kind of adhesive film. Also, it is preferable for the thickness of the adhesive film 5 to be around 1 micron to 30 microns, but it should be noted that even an adhesive film having thickness of more than 30 microns can be applied thereto.

The needle-free injector 4 has a slide housing 41 forming approximately a quadrangular hollow housing. This slide housing 41 is constituted by a front face 41a being faced to the side surface 21a of the base member 2, a right side surface 41b forming a side surface on the right side seen from the front face 41a side, a left side surface 41c forming a side surface on the left side, an upper surface 41d, a lower surface and a back surface.

The right and left side surfaces 41b, 41c of the slide housing 41 are provided with a pair of guide rails 42, 42 projecting downward. These guide rails 42, 42 are engaged with the first guide grooves 221, 221 of the base member 2 slidably, respectively. Thereby, the slide housing 41 is supported by the support board portion 22 of the base member 2 to be movable slidably and is constituted so as to slide on the upper surface 22a of the support board portion 22.

The slide-movement of this slide housing 41 is locked by a locking means which is not shown at two positions of a position at which the slide housing 41 is farthest from the turning control portion 21 and a position at which the front face 41a contacts with the side surface 21a of the turning control portion 21. It is possible for such a locking means to be constituted, for example, by providing a concave portion on one of the lower surface of the slide housing 41 and the upper surface 22a of the support board portion 22 and by providing a convex portion on the other one thereof for being engaged with the concave portion thereof.

Further, there is provided on the upper surface 41d of the slide housing 41 with an operation protrusion portion 43 which projects upward continuously from the long side of the front face side. This operation protrusion portion 43 is provided in order to make the operation to be easy by hooking fingers when slide-operating the slide housing 41.

As shown in FIG. 6, there is provided with the nozzle 44 on the front face 41a of the slide housing 41. This nozzle 44 forms a cylindrical body which projects approximately perpendicularly from the front face 41a of the slide housing 41. The tip of the nozzle 44 is provided with an ejecting opening 441 having a minute diameter. It is preferable for the diameter of the this ejecting opening 441 to be around 0.1 mm to 0.2 mm although it is not limited thereby in particular.

As shown in FIG. 7, the inside of the nozzle 44 is formed by approximately a conical shape in which the diameter thereof becomes smaller toward the tip. Also, the inside of the nozzle 44 forms a liquid room 45 to be filled up with medicinal solution or the like and this liquid room 45 is made to be continuous with the inside of the slide housing 41. In the liquid room 45, liquid is filled up liquid-tightly beforehand. There can be cited for this liquid, for example, remedy for injection or medicinal solution using a macromolecular substance such as hormone, antibody drug, cytokine, vaccine or the like.

Further, there are provided in the inside of the slide housing 41 with a sliding space 46 in communication with the liquid room 45, a piston 47 fitted in the liquid room 45 and the sliding space 46 to be movable slidably, a coil spring 48 showing one embodiment of a high pressure generating means which depresses the piston 47 to the ejecting opening 441 side by high pressure force, and the like.

The piston 47 is constituted by a plunger 471 which is fitted in the liquid room 45 and presses the liquid filled up in the liquid room 45, a flange portion 472 which is continuous with this plunger 471 and also, is fitted in the sliding space 46, and the like. The tip of the plunger 471 is formed in a streamline in which the tip thereof is acuate in order to make the resistance received from the liquid of the liquid room 45 to be small. Thereby, it is possible to depress the liquid of the liquid room 45 at high speeds and it is possible to eject the liquid thereof from the ejecting opening 441 of the nozzle 44 by high pressure force. A compressed coil spring 48 is contacted on the opposite side to the plunger 471 of the flange portion 472 and thereby, the piston 47 is pressurized (biased) toward the ejecting opening 441 side of the nozzle 44.

The foregoing piston 47 is fixed in a state in which the coil spring 48 is compressed by a locking means which is not shown. It is constituted such that this locking means is to be released by pushing down an ejection button 49 provided on the right side surface 41b of the slide housing 41. Then, it is constituted when the locking means is released such that the piston 47 is moved at high speeds by the spring force of the coil spring 48 toward the ejecting opening 441 of the nozzle 44 and the liquid of the liquid room 45 is to be ejected from the ejecting opening 441 of the nozzle 44 by high pressure force. Here, it is needless to say that the eject button 49 can be provided on the left side surface 41c, upper surface 41d or the like of the slide housing 41.

In this exemplified embodiment, a constitution was employed in which the coil spring 48 is used for a high pressure generating means depressing the piston 47 by high pressure force, but it is not limited by the coil spring for the high pressure generating means relating to the present invention and it is possible, for example, to employ a constitution in which a gas such as a high pressure helium gas or the like is used.

It is possible for the material of the base member 2 and the skin deforming member 3 of the foregoing needle-free injection device 1 and the slide housing 41 of the needle-free injector 4 to cite, for example, various kinds of synthetic resins having appropriate strength such as acrylic resin, ABS resin and the like, but it is not limited by this material and it is also possible to use a metal such as aluminum alloy or the like.

Next, it will be explained with respect to the usage (operation) of the needle-free injection device 1 of the first exemplified embodiment.

First, as shown in FIG. 8A, the needle-free injection device 1 is placed on a predetermined position of the body surface F and is pressure-bonded thereon. Thereby, the skin deforming member 3 is adhesively fixed on the body surface F by means of the adhesive film 65.

Next, the second tabular portion 32 of the skin deforming member 3 is depressed toward the first tabular portion 31 side. At that time, the axis portion 222a is to be rotated and the tip of the latch arm 222b is made to be directed to the horizontal direction or upward such that the latch arm 222b of the lock pin 222 mounted on the base member 2 does not interfere with the operation piece 322 of the second tabular portion 32.

Thereby, the depressed second tabular portion 32 is moved so as to be guided by the second guide grooves 223, 223 of the base member 2 and the skin deforming member 3 is bending-deformed by the first hinge portion 35 and the second hinge portion 37. More specifically, the first tabular portion 31 turns upward centering around the hinge axis 61 and at the same time, the coupling portion 33 rises obliquely upward.

In this manner, the bending deformation of the skin deforming member 3 is completed in a state in which the upper surface of the first tabular portion 31 is contacted with the latch slant face 212 of the base member 2 and the skin deforming member 3 becomes in a state as shown in FIG. 8B. At that time, the coupling portion 33 of the skin deforming member 3 is displaced to a position corresponding to that of the opening window 23 on the side surface 21a side of the turning control portion 21 and the upper surface thereof (surface on the opposite side of the surface bonded to the body surface F) becomes the same plane as the side surface 21a of the turning control portion 21.

At the same time, the skin bonded to the first tabular portion 31 of the skin deforming member 3 and the skin bonded to the coupling portion 33 are pulled upward. More specifically, the epidermis E including the stratum corneum SC, the dermis D and the subcutis S are pulled upward respectively and are uplifted. Then, the dermis D or the like of the uplift skin becomes in a state being approximately in parallel with the adhesion surfaces of the first tabular portion 31 and the coupling portion 33. In this state, the layer of the dermis D of the skin bonded to the first tabular portion 31 is to be faced to the ejecting opening 441 which is provided at the nozzle 44 of the needle-free injector 4.

At that time, the length in the width direction of the glue portion 651 of the adhesive film 65 is set to be shorter than the length in the width direction of the skin deforming member 3, so that it is possible to form a predetermined distance between the body surface F bonded to the adhesive film 65 and both the side surfaces of the long sides of the turning control portion 21 and it is possible to uplift the skin without any difficulties. Further, a constitution in which the adhesive film 65 is provided with the wing portions 652, 652 is employed, so that it is possible to form the adhesion surface approximately in a parabolic shape with respect to the body surface F of the uplifted skin and it is possible to prevent the adhesive film 65 from being exfoliated easily from the body surface F.

Next, the axis portion 222a of the lock pin 222 mounted on the base member 2 is rotated and the tip of the latch arm 222b is directed downward. Thereby, the latch arm 222b of the lock pin 222 is engaged with the operation piece 322 of the second tabular portion 32 and the movement of the second tabular portion 32 to the direction departing from the turning control portion 21 is latched. As a result, it is possible to fix the state of the skin which is uplifted by being bonded to the first tabular portion 31 and the coupling portion 33 certainly.

Subsequently, the needle-free injector 4 is depressed toward the turning control portion 21 side. Thereby, the lock by the locking means which is not shown is released and it is possible to slide the needle-free injector 4 toward the turning control portion 21 side.

Thereafter, the needle-free injector 4 is moved slidably along the upper surface 22a of the support board portion 22 and as shown in FIG. 8C, the front face 41a of the needle-free injector 4 is contacted with the side surface 21a of the turning control portion 21. Thereby, the nozzle 44 of the needle-free injector 4 is inserted into the opening window 23 of the base member 2 and the through-hole 331 provided on the coupling portion 33 of the skin deforming member 3 and approaches to the body surface F facing to that through-hole 331. It should be noted in this exemplified embodiment that the gap between the nozzle 44 of the needle-free injector 4 and the body surface F is set to be 0.5 mm or less so as to inject the liquid ejected from the nozzle 44 certainly into the skin.

Also, when the front face 41a of the needle-free injector 4 is contacted with the side surface 21a of the turning control portion 21, the slide-movement of the needle-free injector 4 is to be locked by a locking means which is not shown. Thereby, even if the finger which was depressing the needle-free injector 4 so as to execute the slide-movement thereof is made to be away therefrom, it is possible to maintain the state (state shown in FIG. 8C) in which the ejecting opening 441 of the nozzle 44 is close to the body surface F which can be seen from the through-hole 331 of the skin deforming member 3.

Next, the ejection button 49 of the needle-free injector 4 is depressed. Thereby, the fixation of the piston 47 (see FIG. 7) of the needle-free injector 4 is released and the piston 47 is moved at high speeds toward the ejecting opening 441 of the nozzle 44. Then, the liquid such as the medicinal solution or the like of the liquid room 45 is depressed by the piston 47 and ejected by high pressure force from the ejecting opening 441 of the nozzle 44. As a result, the liquid ejected by high pressure force enters from the body surface F of the skin and is injected into the skin.

At that time, the liquid ejected by high pressure force proceeds in parallel with respect to the direction to which the layer of the dermis D bonded to the first tabular portion 31 of the skin deforming member 3 and uplifted is extended, so that it is possible to inject the liquid into the dermis D efficiently. Also, it happens that an opening having the same minute diameter (preferable, around 0.1 mm to 0.2 mm) as that of the ejecting opening 441 of the needle-free injector 4 is to be opened on the body surface F of the skin by the liquid ejected by high pressure force, so that it is possible to decrease damage of the skin.

Also, the slide-movement of the needle-free injector 4 is locked by the locking means which is not shown and it is fixed in a state in which the ejecting opening 441 of the nozzle 44 is made to approach the body surface F and also is faced to the layer of the dermis D, so that it is possible to inject the liquid ejected from the ejecting opening 441 of the needle-free injector 4 certainly into the dermis D.

After the liquid is ejected by the needle-free injector 4 and the injection into the dermis D terminates and when the needle-free injection device 1 is tried to be detached from the body surface F, first, the lock pin 222 of the support board portion 22 is rotated and the tip of the lock arm 222b is directed upward. Thereby, the engagement between the lock arm 222b and the operation piece 322 provided on the second tabular portion 32 of the skin deforming member 3 is unfastened and the second tabular portion 32 becomes movable slidably to the opposite side with respect to the turning control portion 21.

Subsequently, the second tabular portion 32 of the skin deforming member 3 is slided to the opposite side with respect to the turning control portion 21 and the skin deforming member 3 is returned to be in a tabular shape as shown in FIG. 8A. Thereafter, the skin deforming member 3 is exfoliated from the body surface F and the detachment of the needle-free injection device 1 is accomplished. It should be noted when the skin deforming member 3 is exfoliated from the body surface F that it is possible to accomplish the exfoliation easily if the operation piece 322 of the second tabular portion 22 is pinched by the fingers and uplifted. In addition, it is also possible for the needle-free injection device 1 relating to the present invention to be exfoliated from the body surface F in a state in which the skin deforming member 3 maintains the bending-deformed shape without being returned to a tabular shape.

In the needle-free injection device 1 of this exemplified embodiment, a constitution is employed in which there is provided with the base member 2 mounted with the first tabular portion 31 of the skin deforming member 3 to be revolvable, but it is also possible to employ a constitution for the needle-free injection device relating to the present invention in which there is not provided with the base member 2. In this case, the needle-free injection device is constituted by equipping a skin deforming member, a needle-free injector supported by this skin deforming member to be movable, and the like.

In this needle-free injection device, the skin deforming member is bonded to the body surface F and thereafter, the second tabular portion is depressed toward the first tabular portion side so as to be bending-deformed and the bonded skin is to be uplifted. Next, the nozzle of the needle-free injector is made to approach the body surface F which is seen from the through-hole of the skin deforming member. Thereafter, liquid of a medicinal solution or the like is injected into the dermis D by means of the needle-free injector. It is possible also by such a needle-free injection device to inject liquid into the dermis D efficiently similarly as the needle-free injection device 1 of the first exemplified embodiment.

The needle-free injection device 1A shown in FIG. 9 is a device provided with a cover member 25 covering the nozzle 44 on the base member 2 of the needle-free injection device 1 shown in FIG. 1. Within the constitutions of the this needle-free injection device 1A, common portions as those of the needle-free injection device 1 are put with the same reference numerals and the detailed explanation thereof will be omitted.

The cover member 25 of the needle-free injection device 1A forms approximately a quadrangular frame shape and is formed so as to project approximately perpendicularly by being continuous with the periphery of the opening window 23 on the side surface 21a side. The opposite side with respect to the turning control portion 21 of this cover member 25 is fitted slidably with a groove for the cover member which is not shown and which is provided on a front face 41a of a needle-free injector 4A. Thereby, it is constituted such that the cover member 25 is to be housed in the inside of the needle-free injector 4A when the needle-free injector 4A is slided to the turning control portion 21 side.

According to the foregoing needle-free injection device 1A, the nozzle 44 of the needle-free injector 4A is covered by the cover member 25, so that it is possible to prevent fingers grasping the needle-free injection device 1A from touching the nozzle 44 and it is possible to maintain the ejecting opening 441 of the nozzle 44 to be in a clean condition.

Next, it will be explained with respect to a second exemplified embodiment of a needle-free injection device of the present invention.

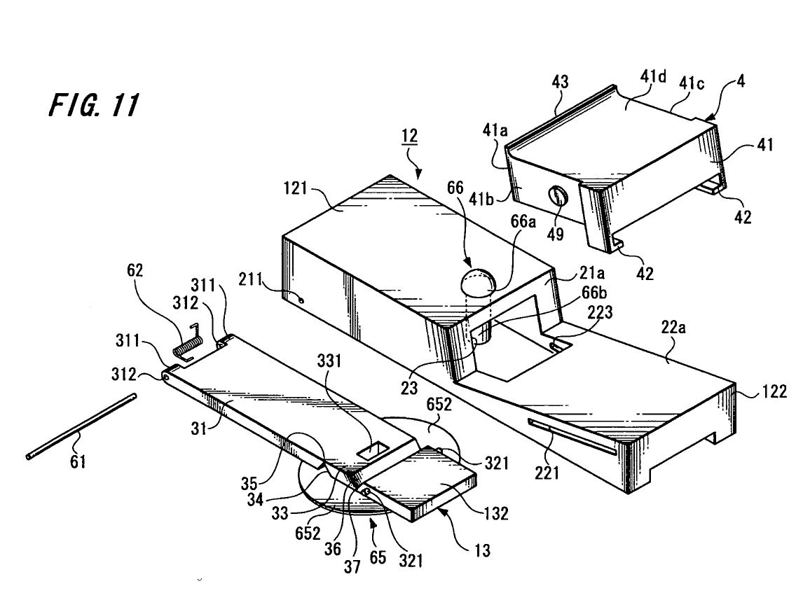

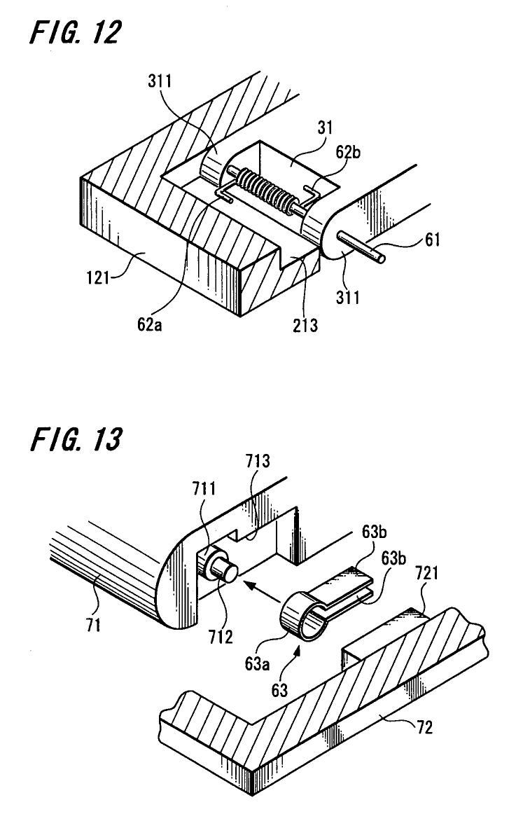

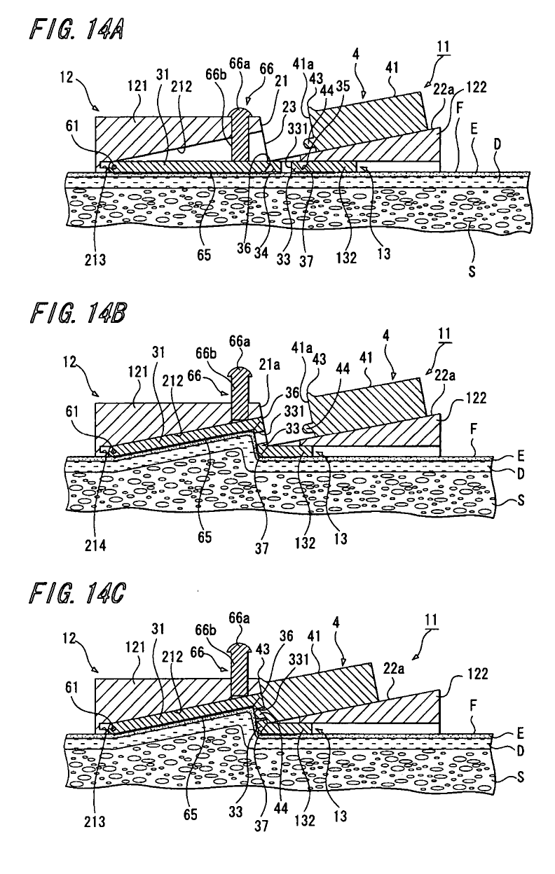

FIG. 10 to FIG. 12 and FIG. 14 show the second exemplified embodiment of the needle-free injection device of the present invention in which FIG. 10 is an external perspective view, FIG. 11 is an exploded perspective view, FIG. 12 is an explanatory diagram showing a spring member, and FIGS. 14A to 14C are cross-section views for explaining the usage of the needle-free injection device shown in FIG. 10. Further, FIG. 13 is an explanatory diagram showing another embodiment of the spring member.

A needle-free injection device 11 showing the second exemplified embodiment of the needle-free injection device of the present invention is different from the needle-free injection device 1 showing the first exemplified embodiment in that a spring member is employed for turning a skin deforming member 13. Within the constitutions of the this needle-free injection device 11, common portions as those of the needle-free injection device 1 are put with the same reference numerals and the detailed explanation thereof will be omitted.

As shown in FIG. 10 and FIG. 11, the needle-free injection device 11 is constituted by equipping a base member 12, a skin deforming member 13 mounted on the base member 12 revolvably, a needle-free injector 4 supported on the base member 12 slidably and the like.

The base member 12 of the needle-free injection device 11 is formed by a turning control portion 121 and a support board portion 122. Also, the skin deforming member 13 of the needle-free injection device 11 is constituted by the first tabular portion 31, the coupling portion 33 and a second tabular portion 132.

The second tabular portion 132 of the skin deforming member 13 is set such that the length thereof extended to the opposite direction of the first tabular portion 31 becomes shorter than that of the second tabular portion 32 of the needle-free injection device 1 showing the first exemplified embodiment. Thereby, it is possible to reduce weight of the second tabular portion 32 and it is possible to make the force demanded for the bending deformation of the skin deforming member 13, which will be described later, to be small.

This skin deforming member 13, similarly as the needle-free injection device 1 showing the first exemplified embodiment, is hinge coupled to the turning control portion 121 by a configuration in which the hinge axis 61 is inserted into the respective bearing openings 312, 312 provided on the pair of bearing pieces 311, 311 of the first tabular portion 31 and into the respective bearing openings 211, 211 of the turning control portion 121. Then, a torsion coil spring 62 intervenes between the pair of bearing pieces 311, 311 of the hinge axis 61.

As shown in FIG. 12, one spring piece 62a of the torsion coil spring 62 is fixed on a spring receiving board 213 of the turning control portion 121 and the other spring piece 62b thereof is fixed on the first tabular portion 31. Thereby, the first tabular portion 31 of the skin deforming member 13 is biased by the spring force of the torsion coil spring 62 so as to turn upward centering around the hinge axis 61.

As shown in FIG. 10 and FIG. 11, the turning control portion 121 of the base member 12 is mounted with a lock member 66 locking the turn of the first tabular portion 31. This lock member 66 is formed by an approximately hemispherical operation button 66a arranged on the upper surface of the turning control portion 121, a latch portion 66b which is continuous with the lower portion of this operation button 66a, and the like.

The operation button 66a of the lock member 66 is fixed on the turning control portion 121 by a lock mechanism which is not shown. Further, the latch portion 66b of the lock member 66 is inserted into a through-hole provided on the upper surface of the turning control portion 121 to be slidable and also, is contacted on the upper surface of the first tabular portion 31 of the skin deforming member 13.

Thereby, the lock member 66 latches the first tabular portion 31 of the skin deforming member 13 so as not to turn upward by the spring force of the coil spring 62 and it is possible to maintain the first tabular portion 31 thereof to be in a state parallel to the horizontal direction. Then, it is constituted such that the lock mechanism, which is not shown, is released by depressing the operation button 66a of the lock member 66 downward and the first tabular portion 31 will turn upward.

The second guide grooves 223, 223 (only one-half thereof is shown in FIG. 11) provided on the support board portion 122 of the base member 12 are provided with lock portions, which are not shown, respectively. It is constituted such that these lock portions are engaged with the pair of guide pins 321, 321 of the second tabular portion 132 which moved to the turning control portion 121 side along with the turn of the first tabular portion 31, respectively. Thereby, the support board portion 122 is constituted so as to fix the second tabular portion 132 which moved to the turning control portion 121 side at the moved position thereof.

It should be noted in this exemplified embodiment that there was employed a constitution in which the torsion coil spring 62 is used for the spring member, but it is also possible, for example, to employ a constitution, as shown in FIG. 13, in which a plate spring 63 is used. This plate spring 63 is constituted by a main body portion 63a forming approximately a cylindrical shape and a pair of arm portions 63b, 63b which are continuous with the side surfaces of this main body portion 63a, and it is constituted such that a spring force is to be operated to the direction of being opened outward.

In this case, a first tabular portion 71 is provided with a fitting portion 711 fitted with the main body portion 63a of the plate spring 63, a turn axis portion 712 continuous with this fitting portion 711, and a spring receiving portion 713 formed by trimming a periphery of the fitting portion 711. Also, the turning control portion 72 is provided with a spring receiving board 721.

Then, one of the arm portions 63b, 63b (upper side in FIG. 13) of the plate spring 63 is fixed on the spring receiving portion 713 of the first tabular portion 71 and at the same time, the other of the arm portions 63b, 63b (lower side in FIG. 13) is fixed on the spring receiving board 721 of the turning control portion 72, whereby the first tabular portion 71 is to be biased so as to turn upward centering around the turn axis portion 712.

Next, it will be explained with respect to the usage (operation) of the needle-free injection device 11 of the second exemplified embodiment.

First, as shown in FIG. 14A, the needle-free injection device 11 is placed on a predetermined position of the body surface F and is pressure-bonded thereon. Thereby, the skin deforming member 13 is adhesively fixed on the body surface F by means of the adhesive film 65.

Next, the operation button 66a of the lock member 66 is depressed downward. Thereby, the lock mechanism, which is not shown, is released and the lock member 66 becomes movable upward. As a result, the first tabular portion 31 of the skin deforming member 13 is turned upward by the spring force of the torsion coil spring 62 centering around the hinge axis 61. Concurrently with this, the coupling portion 33 rises obliquely upward and at the same time, the second tabular portion 132 is moved while it is guided by the guide grooves 223, 223 of the support board portion. As a result, the skin deforming member 13 is bending-deformed by the first hinge portion 35 and the second hinge portion 37.

In this manner, the bending deformation of the skin deforming member 13 is completed in a state in which the upper surface of the first tabular portion 31 is contacted with the latch slant face 212 of the base member 12 and the skin deforming member 13 becomes in a state as shown in FIG. 14B. At that time, the coupling portion 33 of the skin deforming member 13 is displaced to a position corresponding to that of the opening window 23 on the side surface 21a side of the turning control portion 121 and the upper surface thereof (surface on the opposite side of the surface bonded to the body surface F) becomes the same plane as the side surface 21a of the turning control portion 121.

At the same time, the skin bonded to the first tabular portion 31 of the skin deforming member 13 and the skin bonded to the coupling portion 33 are pulled upward. More specifically, the epidermis E including the stratum corneum SC, the dermis D and the subcutis S are pulled upward respectively and are uplifted.

At that time, the dermis D or the like of the uplifted skin becomes in a state being approximately in parallel with the adhesion surfaces of the first tabular portion 31 and the coupling portion 33 and the dermis D of the skin bonded to the first tabular portion 31 becomes in a state in which it is faced to the ejecting opening 441 of the nozzle 44 which is provided at the needle-free injector 4.

Also, in a state in which the bending deformation of the skin deforming member 13 is completed, the pair of guide pins 321, 321 of the second tabular portion 132 are engaged with the lock portions, which is not shown, provided on the second guide grooves 223, 223 of the support board portion 122, respectively. Thereby, the slide-movement of the second tabular portion 132 is latched and it is possible to certainly fix the state of the skin which was bonded to the first tabular portion 31 and the coupling portion 33 and uplifted.

Next, the needle-free injector 4 is moved similarly as the needle-free injection device 1 of the first exemplified embodiment and, as shown in FIG. 14C, the nozzle 44 is made to approach the body surface F facing to the through-hole 331 provided on the coupling portion 33 of the skin deforming member 3. Thereafter, the ejection button 49 of the needle-free injector 4 is depressed and liquid of medicinal solution or the like of the liquid room 45 is made to enter from the body surface F of the skin and is injected into the dermis D.

After the injection of the liquid of medicinal solution or the like terminates and in case of detaching the needle-free injection device 11, similarly as the case of the needle-free injection device 1 of the first exemplified embodiment, the skin deforming member 13 is returned to be in a tabular shape as shown in FIG. 14A and the skin deforming member 13 thereof is exfoliated from the body surface F.

Next, it will be explained with respect to a third exemplified embodiment of a needle-free injection device of the present invention.

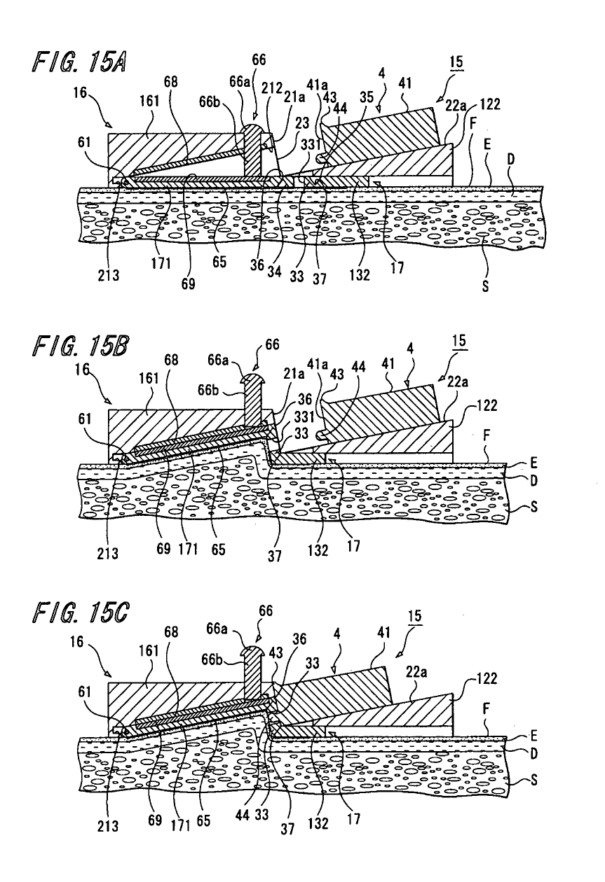

FIGS. 15A to 15C show the third exemplified embodiment of the needle-free injection device of the present invention and are cross-section views for explaining the usage thereof.

A needle-free injection device 15 showing the third exemplified embodiment of the needle-free injection device of the present invention is different from the needle-free injection device 11 showing the second exemplified embodiment in that the skin deforming member 17 is turned by an operation of magnetic force. Within the constitutions of the this needle-free injection device 15, common portions as those of the needle-free injection device 11 are put with the same reference numerals and the detailed explanation thereof will be omitted.

As shown in FIGS. 15A to 15C, the needle-free injection device 15 is constituted by equipping a base member 16, a skin deforming member 17 mounted on the base member 16 revolvably, a needle-free injector 4 supported on the base member 16 to be slidable and the like.

The base member 16 of the needle-free injection device 15 is formed by a turning control portion 161 and a support board portion 122. The turning control portion 161 of this base member 16 is provided with a magnet portion 68. This magnet portion 68 is arranged so as to occupy approximately the whole surface of the latch slant face 212 of the turning control portion 161 and a through-hole is formed at a position corresponding to the lock member 66.

The skin deforming member 17 of the needle-free injection device 15 is constituted by a first tabular portion 171, the coupling portion 33 and the second tabular portion 132. The first tabular portion 171 of the skin deforming member 17 is provided with a magnetic substance 69 for which pulling force toward each other (magnetic force) operates with respect to the magnet portion 68 of the turning control portion 161. This magnetic substance 69 is mounted so as to form an identical plane with the upper surface of the first tabular portion 171 and the size thereof is set to be approximately identical with the plane of the magnet portion 68.

Such a skin deforming member 17, similarly as the skin deforming member 3 of the needle-free injection device 1 showing the first exemplified embodiment, is hinge coupled to the turning control portion 161 by inserting the hinge axis 61 into the respective bearing openings 312, 312 of the first tabular portion 171 and the respective bearing openings 211, 211 of the turning control portion 161. Then, it is constituted such that the first tabular portion 171 of the skin deforming member 17 is turned upward centering around the hinge axis 61 by a pulling force toward each other (magnetic force) which is operated between the magnetic substance 69 and the magnet portion 68 of the turning control portion 161.

Also, the turning control portion 161 of the base member 16 is mounted, similarly as the needle-free injection device 11 showing the second exemplified embodiment, with the lock member 66. Then, this lock member 66 latches the upward turn of the first tabular portion 171 and maintain the first tabular portion 171 thereof to be in a state parallel to the horizontal direction.

Here, it was a constitution in this exemplified embodiment such that the magnet portion 68 is provided on the turning control portion 161 of the base member 16 and the magnetic substance 69 is provided on the first tabular portion 171 of the skin deforming member 17, but it may also be constituted for the needle-free injection device relating to the present invention such that the magnetic substance 69 is provided on the turning control portion 161 and the magnet portion 68 is provided on the first tabular portion 171.

Next, it will be explained with respect to the usage (operation) of the needle-free injection device 15 of the third exemplified embodiment.

First, as shown in FIG. 15A, the needle-free injection device 15 is placed on a predetermined position of the body surface F and is pressure-attached thereon. Thereby, the skin deforming member 17 is adhesively fixed on the body surface F by means of the adhesive film 65.

Next, the operation button 66a of the lock member 66 is depressed downward. Thereby, the lock mechanism, which is not shown, is released and the lock member 66 becomes movable upward. As a result, the first tabular portion 171 of the skin deforming member 17 is turned upward by the magnetic force operated between the magnet portion 68 and the magnetic substance 69 of the turning control portion 161 centering around the hinge axis 61. Concurrently with this, the coupling portion 33 rises obliquely upward and at the same time, the second tabular portion 132 is moved while it is guided by the guide grooves 223, 223 of the support board portion. As a result, the skin deforming member 17 is bending-deformed at the first hinge portion 35 and the second hinge portion 37.

In this manner, the bending deformation of the skin deforming member 13 is completed in a state in which the magnet portion 68 of the first tabular portion 171 is contacted with the magnetic substance 69 of the turning control portion 161 and the skin deforming member 17 becomes in a state as shown in FIG. 15B. At that time, the coupling portion 33 of the skin deforming member 17 is displaced to a position corresponding to that of the opening window 23 on the side surface 21a side of the turning control portion 161 and the upper surface thereof (surface on the opposite side of the surface bonded to the body surface F) becomes the same plane as the side surface 21a of the turning control portion 161.

At the same time, the skin bonded to the first tabular portion 171 of the skin deforming member 17 and the skin bonded to the coupling portion 33 are pulled upward. More specifically, the epidermis E including the stratum corneum SC, the dermis D and the subcutis S are pulled upward respectively and are uplifted.

At that time, the dermis D or the like of the uplifted skin becomes in a state being approximately in parallel with the adhesion surfaces of the first tabular portion 171 and the coupling portion 33 and the dermis D of the skin bonded to the first tabular portion 171 becomes in a state in which it is faced to the ejecting opening 441 of the nozzle 44 which is provided at the needle-free injector 4.

Thereafter, by performing similar procedures as those for the needle-free injection device 11 of the second exemplified embodiment, the liquid of medicinal solution or the like filled up in the liquid room 45 of the needle-free injector 4 is made to enter from the body surface F of the skin and is injected into the dermis D. Also in a case in which the needle-free injection device 15 is detached, it is accomplished, similarly as the case of the needle-free injection device 11, by returning the skin deforming member 17 to be in a tabular shape, as shown in FIG. 15A, and by exfoliating the skin deforming member 17 from the body surface F.

Next, it will be explained with respect to a fourth exemplified embodiment of a needle-free injection device of the present invention.

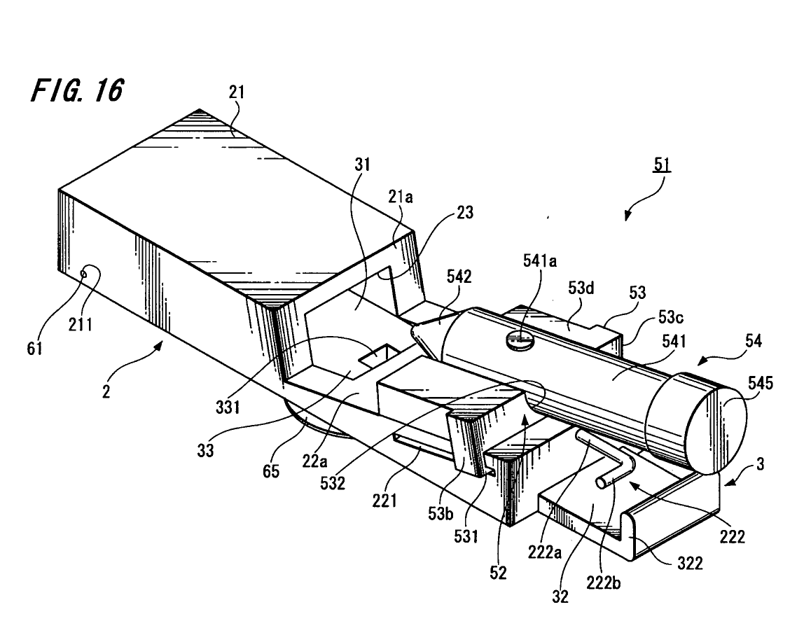

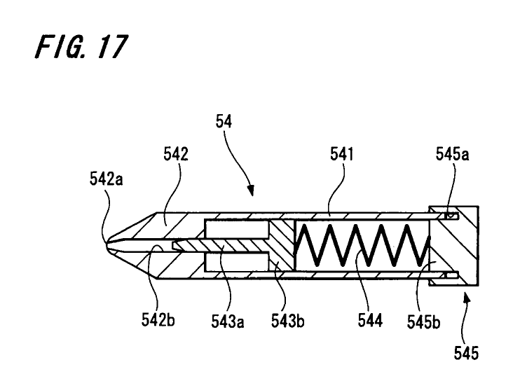

FIG. 16 and FIG. 17 show the fourth exemplified embodiment of the needle-free injection device of the present invention in which FIG. 16 is an external perspective view and FIG. 17 is a cross-section view indicating the inside of the injector body shown in FIG. 16 schematically.

A needle-free injection device 51 showing the fourth exemplified embodiment of the needle-free injection device of the present invention is different from the needle-free injection device 1 showing the first exemplified embodiment in a needle-free injector 52 supported by the base member 2 to be slidable. Within the constitutions of this needle-free injection device 51, common portions as those of the needle-free injection device 1 are put with the same reference numerals and the detailed explanation thereof will be omitted.

As shown in FIG. 16, the needle-free injector 52 of the needle-free injection device 51 is constituted by equipping a slide member 53 supported by the base member 2 to be slidable, an injector body 54 of approximately a cylindrical shape fixed on this slide member 53 and the like.

The slide member 53 of the needle-free injector 52 forms an elongated rectangular solid and is constituted by a front face 53a faced to the side surface 21a of the base member 2, a right side surface 53b forming a side surface on the right side seen from the front face 53a side, a left side surface 53c forming a side surface on the left side, an upper surface 53d, a lower surface, and a back surface.

There are provided on the side surfaces 53b, 53c of the right and left sides of the slide member 53 with a pair of guide rails 531, 531 (only the right side surface 53b is shown in FIG. 16) which project downward. These guide rails 531, 531 are engaged with the first guide grooves 221, 221 of the base member 2 to be slidable, respectively. Thereby, the slide member 53 is constituted so as to be supported by the support board portion 22 of the base member 2 to be slidable and so as to slide on the upper surface 22a of the support board portion 22.

The slide-movement of this slide member 53 is constituted such that the slide member 53 is to be locked by a locking means which is not shown at two positions of the farthest position from the turning control portion 21 and a position at which the front face 53a contacts with the side surface 21a of the turning control portion 21. It is possible for such a locking means to be constituted, similarly as the locking means provided on the needle-free injection device 1 of the first exemplified embodiment, by providing a concave portion on one of the lower surface of the slide member 53 and the upper surface 22a of the support board portion 22 and by providing a convex portion on the other one thereof for being engaged with the concave portion thereof.

Also, the upper surface 53d of the slide member 53 is provided with a concave portion 532 for fixation for fixing the injector body 54. This concave portion 532 for fixation is formed such that the cross-sectional shape thereof is in approximately a half-round shape so as to fit the outer shape of the injector body 54 and the injector body 54 is firmly fixed on this concave portion 532 for fixation by a fixation means such as an adhesive agent or the like.

As shown in FIG. 17, the injector body 54 of the needle-free injector 52 is constituted by equipping an outer cylinder 541 of approximately a cylindrical shape, a nozzle 542 continuous with one terminal of this outer cylinder 541, a piston 543 pressure-transporting the liquid filled up in the nozzle to the tip of the nozzle thereof, a coil spring 544 showing one embodiment of a high pressure generating means which presses the piston 543 with high pressure force to the nozzle 542 side, a compressing knob 545 for compressing the coil spring 544 and the like. There is provided at the edge portion on the opposite side of the nozzle 542 of the outer cylinder 541 with a male screw portion, which is not shown, along the circumference surface thereof and a female screw portion of the compressing knob 545, which will be described later, is screwed on this male screw portion.

The nozzle 542 of the injector body 54 is formed by approximately a conical shape in which the diameter thereof becomes smaller toward the tip. The tip of this nozzle 542 is provided with an ejecting opening 542a having a minute diameter and the inside thereof is provided with a liquid room 542b in communication with the ejecting opening 542a. It is preferable for the diameter of the ejecting opening 542a of the nozzle 542 to be around 0.1 mm to 0.2 mm although it is not limited thereby in particular.

The liquid room 542b of the nozzle 542 is formed by approximately a conical shape in which the diameter thereof becomes smaller along with the approach to the ejecting opening 542a side. Thereby, it is possible when the liquid filled up in the liquid room 542b is ejected from the ejecting opening 542a to prevent or reduce the loss of the water pressure thereof. There can be cited for the liquid filled up in the liquid room 542b, similarly as that of the first exemplified embodiment, remedy for injection or medicinal solution using a macromolecular substance such as hormone, antibody drug, cytokine, vaccine or the like.

The piston 543 is constituted by a plunger 543a fitted slidably in the liquid room 542b of the nozzle 542, a flange portion 543b continuous with this plunger 543a and also fitted slidably in the inside of the outer cylinder 541, and the like. The tip of the plunger 543a is formed in a streamline in which the tip thereof is acuate in order to make the resistance received from the liquid of the liquid room 542b to be small. Thereby, it is possible to pressure-transport the liquid of the liquid room 542b at high speeds and it is possible to eject the pressure-transported liquid from the ejecting opening 542a of the nozzle 44 by high pressure force. One terminal of a coil spring 544 is fixed on the opposite side of the plunger 543a of the flange portion 543b.

Such a piston 543 is fixed by a locking means, which is not shown, at a position by which the liquid in the liquid room 542b will not be pushed out from the ejecting opening 542a of the nozzle 542. It is constituted such that this locking means is to be released by pushing down an ejection button 541a (see FIG. 16) provided on the outer surface of the outer cylinder 541.

The compressing knob 545 forms approximately a cylinder body and includes a circular slit 545a provided on the surface facing to the outer cylinder 541 and a press portion 545b formed in the inside of this circular slit 545a. The circular slit 545a of the compressing knob 545 is provided with a female screw portion, which is not shown, corresponding to a male screw portion provided at the edge portion of the outer cylinder 541 and thereby, it is constituted such that the compressing knob 545 is to be screwed on the edge portion of the outer cylinder 541. The press portion 545b is inserted into the inside of the outer cylinder 541 when the compressing knob 545 is screwed on the edge portion of the outer cylinder 541. One terminal of the coil spring 544 is fixed on this press portion 545b.

According to the injector body 54 having the foregoing constitution, it is possible to eject the liquid filled up in the liquid room 542b from the ejecting opening 542a along the following procedure. First, the compressing knob 545 is rotated so as to be screwed on the edge portion of the outer cylinder 541. Thereby, the press portion 545b of the compressing knob 545 inserted into the inside of the outer cylinder 541 is displaced to the piston 543 side and the coil spring 544 is compressed.

Next, the ejection button 541a is depressed and the locking means of the piston 543 is released. Thereby, the piston 543 is pressed to the ejecting opening 542a side of the nozzle 542 by the spring force of the coil spring 544, and the liquid filled up in the liquid room 542b is pressure-transported and ejected by high pressure force from the ejecting opening 542a.

According also to the needle-free injection device 51 having the constitution as described above, similarly as the needle-free injection device 1 showing the first exemplified embodiment, it is possible to make the layer of the dermis D to face to the ejecting opening 542a of the needle-free injector 52 by uplifting the skin bonded on the first tabular portion 31 and the coupling portion 33 of the skin deforming member 3 and it is possible to inject the liquid of the medicinal solution or the like ejected by high pressure force from the ejecting opening 542a thereof certainly into the dermis D.

In this exemplified embodiment, the needle-free injector 52 is constituted integrally by fixing the injector body 54 on the slide member 53, but it is also possible to employ a constitution in which the injector body 54 is mounted detachably with respect to the slide member 53. In this case, it is possible to store the needle-free injection devices 51 on which the injector bodies 54 are not mounted by piling them, so that it is possible to attempt space-saving of the storage area.

In addition, it is allowed for the injector body 54 to be constituted by an ejecting device equipping the nozzle 542, the piston 543 and the like and a high pressure generator which is mounted detachably on the ejecting device and also equips the coil spring 545, the compressing knob 546 and the like, in which the ejecting device is to be fixed on the slide member 53. In this case, it is possible to use the high pressure generator repeatedly with respect to a plurality of needle-free injection devices and it is possible to attempt cost reduction by reducing the number of parts of the needle-free injection device.

Next, it will be explained with respect to a fifth exemplified embodiment of a needle-free injection device of the present invention.

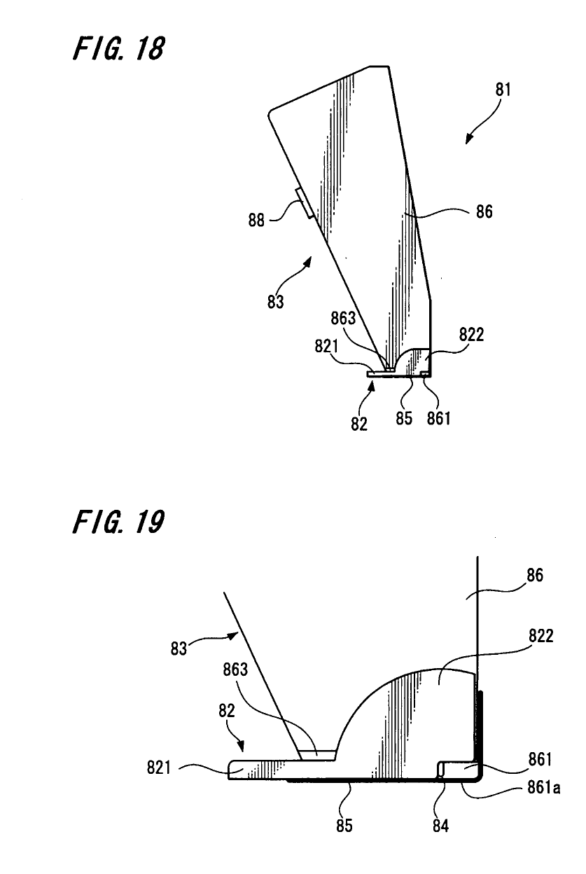

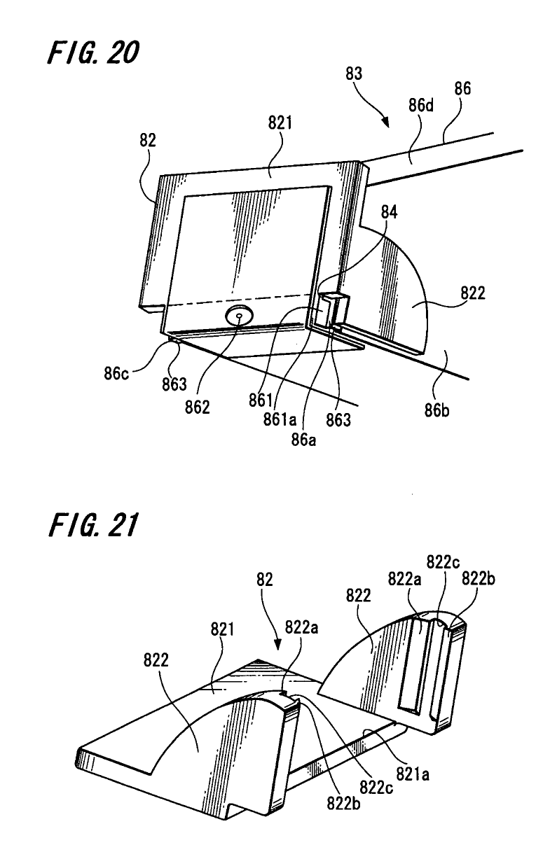

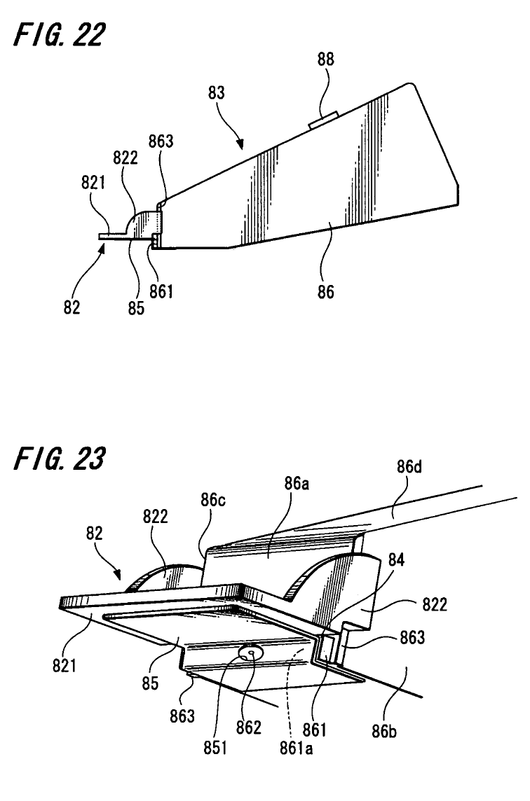

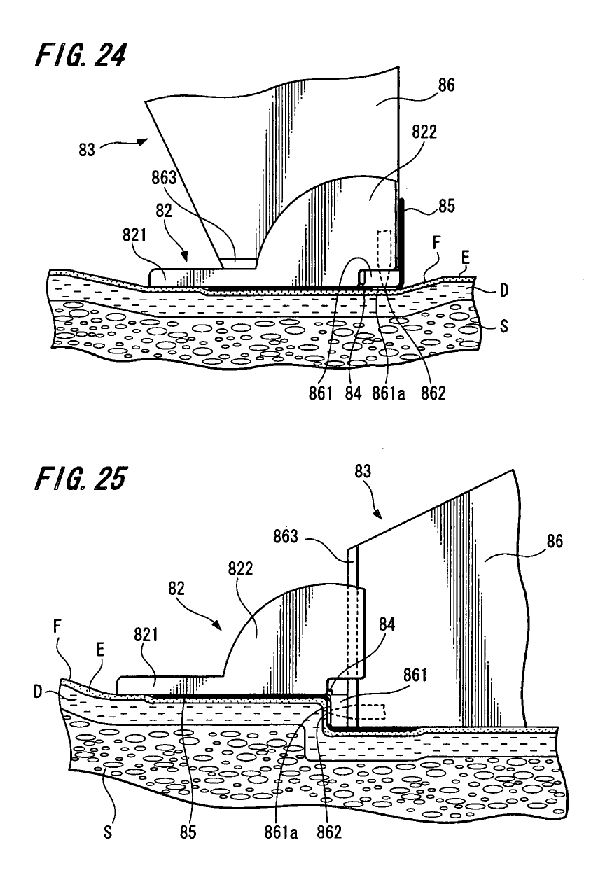

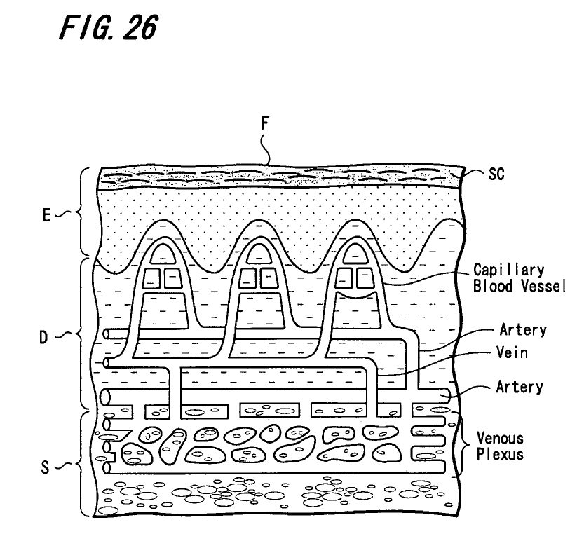

FIG. 18 to FIG. 25 show the fifth exemplified embodiment of the needle-free injection device of the present invention in which FIG. 18 is a side elevational view of a needle-free injection device, FIG. 19 is an enlarged view obtained by enlarging a main portion of the needle-free injection device shown in FIG. 18, FIG. 20 is an explanatory diagram seeing the main portion shown in FIG. 19 from the lower side, FIG. 21 is an explanatory diagram explaining a positioning portion of the needle-free injection device, FIG. 22 is a side elevational view of a state in which the needle-free injector is turned approximately by 90 degree with respect to the positioning portion, FIG. 23 is an explanatory diagram seeing the state shown in FIG. 22 from the lower side thereof, and FIG. 24 and FIG. 25 are cross-section views explaining the usage of the needle-free injection device shown in FIG. 18.

The needle-free injection device 81 showing the fifth exemplified embodiment of the needle-free injection device of the present invention is constituted by equipping a positioning portion 82, a needle-free injector 83, a hinge portion 84 interlinking the positioning portion 82 and the needle-free injector 83 to be revolvable, an adhesive film 85 showing one embodiment of an adhesion member for fixing the positioning portion 82 and a portion of the needle-free injector 83 on the body surface F, and the like. It should be noted that a stratum corneum SC, an epidermis E including the stratum corneum SC, a dermis D, and a subcutis S are arranged in a form of laminae on the lower side of the body surface F on which the positioning portion 82 and the portion of the needle-free injector 83 is fixed (see FIG. 24 or the like).

As shown in FIG. 18 to FIG. 21 or the like, the positioning portion 82 of the needle-free injection device 81 is constituted by a main body plate 821 forming approximately a rectangular plate body, a pair of rising pieces 822, 822 rising approximately perpendicularly on two short sides of this main body plate 821 continuously, respectively, and the like. One of the long sides 821a of the main body plate 821 is interlinked to the needle-free injector 83 through the hinge portion 84. Also, the surface on the opposite side with respect to the surface with which the pair of rising pieces 822, 822 of the main body plate 821 are continuous is made to be a plane for fixing the positioning portion 82 by being appressed to the body surface F.

As shown in FIG. 21, the pair of rising pieces 822, 822 of the positioning portion 82 form approximately fan-shaped plate bodies respectively and the respective corner portions thereof are projected outward from the one of the long sides 821a of the main body plate 821. There are provided on the surfaces facing to each other of these pair of rising pieces 822, 822 with first protrusion portions 822a extended approximately perpendicularly with respect to the main body plate 821 and second protrusion portions 822b respectively. Thereby, engagement grooves 822c are formed between the two protrusion portions 822a, 822b of the respective rising pieces 822.

The respective engagement grooves 822c, 822c of the pair of rising pieces 822, 822 are engaged with engagement protrusion portions 863, 863 of the needle-free injector 83, which will be described later. At that time, the respective protrusion portions 822a, 822a are formed with slant planes such that the engagement protrusion portions 863, 863 of the needle-free injector 83 can easily climb over the first protrusion portions 822a, 822a of the pair of rising pieces 822, 822.

Here, when the engagement protrusion portions 863, 863 of the needle-free injector 83 climb over the first protrusion portions 822a, 822a of the rising pieces 822, 822, it is constituted such that the rising pieces 822, 822 are to be bended outward, respectively.

As shown in FIG. 18, FIG. 20 or the like, the needle-free injector 83 has a turn housing 86 forming an elongated housing having a space inside. This turn housing 86 is constituted by a front face 86a interlinked to the positioning portion 82 through the hinge portion 84, a right side surface 86b forming a side surface on the right side seen from the front face 86a side, a left side surface 86c forming a side surface on the left side, an upper surface 86d, a lower surface and a back face.

The front face 86a of the turn housing 86 is provided with an interlinked protrusion portion 861 showing one embodiment of a coupling portion interlinked to the hinge portion 84. This interlinked protrusion portion 861 forms an elongated rectangular solid and one surface thereof (lower surface) is made to be the same surface of the lower surface of the turn housing 86. Then, the front face of the interlinked protrusion portion 861 is made to be a press surface 861a for depressing and uplifting the skin.

The press surface 861a of the interlinked protrusion portion 861 is provided, as shown in FIG. 20 or the like, with an ejecting opening 862 from which liquid of medicinal solution or the like is to be ejected by high pressure force. More specifically, the interlinked protrusion portion 861 has a role as a nozzle of the needle-free injector 83. It is preferable for the diameter of the ejecting opening 862 provided on the interlinked protrusion portion 861 to be around 0.1 mm to 0.2 mm although it is not limited thereby in particular.

The inside of the turn housing 86 is provided, similarly as that of the slide housing 41 of the first exemplified embodiment, with a liquid room in communication with the ejecting opening 862, a piston pressing liquid of medicinal solution or the like filled up in the liquid room to the ejecting opening 862 side, a coil spring showing one embodiment of the high pressure generating means pressing the piston thereof by high pressure force, and the like. Then, it is constituted by pushing down an ejection button 88 provided on the upper surface 86d of the turn housing 86 such that the piston is moved at high speeds by the spring force of the coil spring toward the ejecting opening 862 and the liquid of the liquid room is to be ejected by high pressure force from the ejecting opening 862.

The right and left side surfaces 86b, 86c of the turn housing 86 are provided with engagement protrusion portions 863, 863 projecting approximately perpendicularly, respectively. These engagement protrusion portions 863, 863 are engaged with a pair of engagement grooves 822c, 822c of the positioning portion 82. Thereby, it is constituted as shown in FIG. 23 or the like such that the needle-free injection device 81 is fixed in a state in which the front face 86a of the needle-free injector 83 becomes approximately perpendicular with respect to the main body plate 821 of the positioning portion 82.

The hinge portion 84 is set to have low stiffness compared with that of the positioning portion 82 or the turn housing 86 of the needle-free injector 83. Thereby, the needle-free injector 83 is constituted so as to be revolvable within the angular range of approximately 90 degree from a state in which the front face 86a is contacted to the upper surface of the positioning portion 82 (state shown in FIG. 18 and FIG. 20) until a state in which the front face 86a of the needle-free injector 83 becomes approximately perpendicular with respect to the upper surface of the positioning portion 82 (state shown in FIG. 22 and FIG. 23).

It is possible for the material of the foregoing turn housing 86, the positioning portion 82 and the hinge portion 84 to cite, for example, an ABS (acrylonitrile-butadiene-styrene resin). However, the material of the base member 2 is not limited by the ABS and it is needless to say that synthetic resins of engineering plastic or others can be applied thereto and it is also possible to use metals such as aluminum alloy or the like other than the synthetic resins.

As shown in FIG. 19, FIG. 20, FIG. 23 and the like, the adhesive film 85 is formed approximately in a rectangular sheet shape and is glued so as to cover the lower surface of the positioning portion 82, the hinge portion 84, the press surface 861a of the interlinked protrusion portion 861 and the lower surface on the interlinked protrusion portion 861 side of the turn housing 86. This adhesive film 85 is provided with an opening hole 851 for exposing the ejecting opening 862 provided on the interlinked protrusion portion 861.

It should be noted that an exfoliating paper, which is not shown, is attached on the surface of the adhesive film 85 which is bonded to the body surface F until just before the needle-free injection device 81 is used. The opening hole 851 of the adhesive film 85 is sealed by means of this exfoliating paper, so that it is made to be a state in which the liquid of medicinal solution or the like is not to leak from the ejecting opening 862 exposed through the opening hole 851.

It is possible for the material of the adhesive film 85 to cite polyethylene terephthalate (PET) and polyethylene which do not expand or contract and the like, but it is not limited by this and it is possible to use various kinds of materials such as polyurethane and others which are used as this kind of adhesive film. Also, it is preferable for the thickness of the adhesive film 85 to be around 1 micron to 30 microns, but it should be noted that even an adhesive film having thickness of more than 30 microns can be applied thereto.

Next, it will be explained with respect to the usage (operation) of the needle-free injection device 81 of the fifth exemplified embodiment.

First, as shown in FIG. 24, the needle-free injection device 81 from which the exfoliating paper of the adhesive film 85 is exfoliated beforehand is mounted and pressure-attached onto a predetermined position of the body surface F. Thereby, the positioning portion 82 of the needle-free injection device 81 and the press surface 861a of the needle-free injector 83 are adhesively fixed on the body surface F by means of the adhesive film 85.

Next, as shown in FIG. 25, the needle-free injector 83 is turned approximately by 90 degree to the body surface F side. At that time, the engagement protrusion portions 863, 863 of the needle-free injector 83 are to be engaged with the pair of engagement grooves 822a, 822a provided on the rising pieces 822, 822 of the positioning portion 82. Thereby, the needle-free injection device 81 is fixed in a state in which the needle-free injector 83 is turned approximately by 90 degree to the body surface F side.

At that time, the press surface 861a of the interlinked protrusion portion 861 which is provided on the needle-free injector 83 becomes in an approximately perpendicular state with respect to the positioning portion 82 and the skin bonded to the press surface 861a of the interlinked protrusion portion 861 is depressed. Thereby, the press surface 861a of the interlinked protrusion portion 861 and the skin bonded to the positioning portion 82 are pulled upward. More specifically, the epidermis E including the stratum corneum SC, the dermis D and the subcutis S are uplifted respectively by being pulled upward.

At the same time, the lower surface of the needle-free injector 83 is adhesively fixed on the body surface F by the adhesive film 85. Thereby, there is formed a step between the skin bonded to the positioning portion 82 and the skin bonded or appressed to the lower surface of the needle-free injector 83. In this state, the layer of the dermis D of the skin bonded to the positioning portion 82 is made to face to the ejecting opening 862 provided on the interlinked protrusion portion 861 of the needle-free injector 83. Then, the ejecting opening 862 of the interlinked protrusion portion 861 approaches with respect to the body surface F bonded to the interlinked protrusion portion 861 by a distance of as much as the thickness of the adhesive film 85.

Next, the ejection button 88 of the needle-free injector 83 is depressed. Thereby, the piston of the needle-free injector 83 is moved at high speeds toward the ejecting opening 862. Then, the liquid of the medicinal solution or the like in the liquid room is pressed by the piston and ejected by high pressure force from the ejecting opening 862. As a result, the liquid ejected by high pressure force enters from the body surface F of the skin and is injected into the skin.

At that time, the liquid ejected by high pressure force proceeds in parallel with respect to the direction to which the layer of the dermis D bonded to the positioning portion 82 and uplifted is extended, so that it is possible to inject the liquid into the dermis D efficiently. Also, the posture of the needle-free injection device 81 is fixed in a state in which the ejecting opening 862 of the needle-free injector 83 faces to the layer of the uplifted dermis D by engaging the engagement protrusion portions 863, 863 of the needle-free injector 83 with the pair of engagement grooves 822a, 822a of the positioning portion 82, so that it is possible to inject the liquid of the medicinal solution or the like ejected from the ejecting opening 862 certainly into the dermis D.

As explained above, according to the needle-free injection device of the present invention, liquid of medicinal solution or the like is ejected by a needle-free injector to the direction to which a predetermined layer of a skin, for example, a dermis is extended after uplifting the skin by a skin deforming member, so that it is possible to inject the liquid thereof efficiently and certainly into the dermis.

It should be noted that the above-described exemplified embodiments were explained by using examples in which liquid of medicinal solution or the like is always injected into the dermis D, but it is possible to use the needle-free injection device of the present invention also in case of injecting liquid of medicinal solution or the like into a intracutaneous area, a subcutis or further a muscle other than the dermis.

It should be noted for the skin deforming means of the present invention that it is not limited by the aforesaid exemplified embodiments and it is possible to cite configurations in which a predetermined portion of the skin is gathered and lifted by pushing the both sides thereof, the predetermined portion of the skin is hooked and lifted and the like.

Also, the needle-free injection device of the present invention is not limited by the above-mentioned each exemplified embodiment and besides that, various modifications or changes can be employed for the materials, the constitutions or the like in the region without departing from the configuration of the present invention. For example, a constitution was employed in the aforesaid exemplified embodiments in which the skin deforming member and the positioning portion or the like are adhesively fixed on the skin by the adhesion member, but another constitution may be employed in which a sucking means is provided on the skin deforming member and the positioning portion or the like and by sucking the skin using this sucking means, the skin is to be fixed on the skin deforming member and the positioning portion or the like.

Having described preferred embodiments of the invention with reference to the accompanying drawings, it is to be understood that the invention is not limited to those precise embodiments and that various changes and modifications could be effected therein by one skilled in the art without departing from the spirit or scope of the invention as defined in the appended claims.

What is claimed is:

1. A needle-free injection device comprising:
a skin deforming member for deforming a skin; and
a needle-free injector ejecting liquid by high pressure with respect to the skin deformed by said skin deforming member and injecting said liquid into said skin.

2. The needle-free injection device according to claim 1, wherein said skin deforming member has two or more planes fixed on the skin and the skin is deformed so as to form a step portion by said two or more planes.

3. The needle-free injection device according to claim 2, wherein it is constituted such that said needle-free injector ejects said liquid approximately parallel to the any one of said two or more planes of said skin deforming member and injects it into said step portion.

4. A needle-free injection device comprising:
a needle-free injector ejecting filled up liquid by high pressure;
a base member supporting said needle-free injector to be movable;
a skin deforming member mounted on said base member and also deforming the skin, wherein
it is constituted such that said needle-free injector is moved so as to approach to the skin deformed by said skin deforming member and the liquid is ejected by high pressure and injected with respect to said deformed skin.

5. The needle-free injection device according to claim 4, wherein said needle-free injector comprises a slide member supported on said base portion to be movable, and an injector body mounted on said slide member detachably.

6. The needle-free injection device according to claim 4, wherein said skin deforming member comprises:
a first tabular portion supported on said base member to be revolvable and also having a plane fixed on the skin;
a coupling portion connected to said first tabular portion through a first hinge portion and also having a plane fixed on the skin; and
a second tabular portion connected to said coupling portion through a second hinge portion and also having a plane fixed on the skin, wherein
the skin is deformed so as to form a step portion between said first tabular portion and said second tabular portion by bending-deforming said skin deforming member.

7. The needle-free injection device according to claim 6, wherein said first hinge portion is provided by forming a V-shaped groove to be opened downward at a connection portion of said first tabular portion and said coupling portion and also, said second hinge portion is provided by forming a V-shaped groove to be opened upward at a connection portion of said second tabular portion and said coupling portion.

8. The needle-free injection device according to claim 6, wherein said skin deforming member is bending-deformed by depressing said second tabular portion to said first tabular portion side so as to turn said first tabular portion upward.

9. The needle-free injection device according to claim 6, wherein there is provided with a spring member biasing said first tabular portion upward and said first tabular portion is turned by the spring force of said spring member so as to bending-deform said skin deforming member.

10. The needle-free injection device according to claim 6, wherein there is provided with a magnet portion on either one of said first tabular portion or said base member and also there is provided with a magnetic portion on the other one thereof which pulls said magnet portion toward each other, and said first tabular portion is made to turn by the magnetic force operated between said magnet portion and said magnetic portion so as to bending-deform said skin deforming member.

11. A needle-free injection device comprising
a positioning portion having a plane fixed on a skin;
a coupling portion connected to said positioning portion to be revolvable through a hinge portion and also having a plane fixed on the skin; and
a needle-free injector constituted integrally with said coupling portion and also being provided with an ejecting opening on a plane fixed on the skin of said coupling portion, wherein
it is constituted such that the skin fixed on said positioning portion and said coupling portion is uplifted by changing posture of said needle-free injector and by turning said coupling portion with respect to said positioning portion, and liquid is ejected from said ejecting opening by high pressure and injected into said uplifted skin.

12. The needle-free injection device according to claim 11, wherein when said coupling portion is turned with respect to said positioning portion, the plane fixed on the skin at said coupling portion becomes approximately perpendicular with respect to the plane fixed on the skin of said positioning portion.

13. The needle-free injection device according to claim 11, wherein there is provided with an engagement projection portion on one of said positioning portion and said needle-free injector and also there is provided with an engagement concave portion on the other one thereof which is to be engaged with said engagement projection portion when said coupling portion is turned with respect to said positioning portion, and it is constituted such that a state in which said coupling portion is turned with respect to said positioning portion is to be maintained.
